(19) United States
(12) Patent Application Publication
(10) Pub. No.: US 2019/0048274 A1
(43) Pub. Date: Feb. 14, 2019

(54) PROCESS FOR THE PRODUCTION OF ONE OR MORE PRODUCT FUELS

(71) Applicant: JOHNSON MATTHEY DAVY TECHNOLOGIES LIMITED, London (GB)

(72) Inventors: Jason F. GILES, Missouri City, TX (US); George R. PHILLIPS, South Charleston, WV (US); Martin Lucas SMIDT, London (GB); Stephane WAMBERGUE, London (GB)

(21) Appl. No.: 16/077,109

(22) PCT Filed: Feb. 2, 2017
(86) PCT No.: PCT/GB2017/050258

§ 371 Date: Aug. 10, 2018

(30) Foreign Application Priority Data
Feb. 11, 2016 (GB) 1602497.8

(51) Int. Cl. C10L 1/02; C07C 45/50; C07C 29/141

(52) U.S. Cl. CPC C10L 1/023; C07C 45/50; C07C 47/02; C10L 2200/0423; C10L 2270/023; C07C 29/141

(57) ABSTRACT

A process for the production of one or more product fuels, particularly product motor fuels comprising: recovering a stream comprising olefin from one or more processing units within an oil refinery; feeding said olefin stream to a hydroformylation zone within the refinery; contacting said olefin stream with carbon monoxide and hydrogen in the presence of a hydroformylation catalyst and operating said hydroformylation zone under hydroformylation conditions such that at least a portion of the olefin is converted to an aldehyde having an additional carbon atom to the olefin; recovering a stream comprising said aldehyde from the hydroformylation zone and passing said aldehyde stream to a hydrogenation zone operated under hydrogenation conditions such that at least a portion of the aldehyde is converted to the corresponding alcohol; and recovering a stream comprising alcohol and forwarding the alcohol to one or more product fuel pools, preferably product motor fuel pools within the refinery.

The present invention relates to a process for the production of one or more product fuels, such as product motor fuels. In particular, it relates to a process for the production of one or more product fuels, such as product motor fuels, within an oil refinery.

As is well understood, an oil refinery is a plant where crude oil is processed and refined into useful products many of which are product fuels such as product motor fuels. Further detail of those useful product fuels is discussed in below. However, it will be understood that product motor fuels includes those fuels which are suitable for use in motors such as automobiles, lorries, motorbikes, and also marine engines. In one arrangement, aviation fuels may fall within this definition.

In addition, to these product fuels, various compounds having a low number of carbons are produced which are not useful as product fuels and which are therefore waste product or are simply burnt to provide energy for the refinery. As indicated above, when crude oil is processed in an oil refinery a variety of useful products are formed. The refinery therefore comprises a plurality of processing units appropriate for the production of the various desired products. Some of the desired products are product fuels. This will include transportation fuels such as gasoline, diesel, aviation fuel and the like.

These fuels generally comprise a mixture of hydrocarbon compounds. For example, the bulk of a typical gasoline comprises C₄ to C₁₂ hydrocarbons and may be a mixture of alkanes, naphthenes and aromatics. The ratio of the various compounds present in the finished product will depend on the processing units present in a particular refinery, the composition of the crude oil feed to the refinery and the grade of gasoline required, in particular, the desired octane rating. Similarly, the bulk of diesel will comprise C₈ to C₂₁ hydrocarbon compounds.

There are generally two kinds of aviation fuel, there is jet fuel and aviation gasoline although jet fuel is more commonly used and therefore is produced in larger volumes. Jet fuel generally comprises kerosene. Kerosene, which is also used as a heating, cooking and lighting fuel, generally comprises C₆ to C₁₆ hydrocarbon compounds.

The octane rating, which is also known as the octane number, is a standard measure of the gasoline performance. The higher the octane number, the more compression the fuel can withstand before detonating. The octane rating is defined by comparison with a mixture of iso-octane and heptane. The octane rating of fuel may be increased by the inclusion of additives not produced within the refinery. For example, ethanol may be blended into the fuel to increase the octane number.

Since a particular fuel produced in a refinery will comprises a mixture of compounds which may have been gathered from one or more processing units within the refinery, the fuel can be referred to as a particular fuel ‘pool’. Thus, for example, a gasoline pool may be formed by the hydrocarbons produced having the desired hydrocarbon profile.

It will be understood that some compounds produced in the refinery may be suitable to be added to more than one pool. This enables the operator of the refinery to tune the streams supplied to the various pools to achieve the desired profile for a particular fuel requirement, such as a desired octane number.

Generally more than one fuel pool will be formed thus, for example, a refinery may comprise means for forming one or more of a gasoline pool, a diesel pool, an aviation fuel pool, and a kerosene pool.

As the drive to greener fuels continues, fuels derived from non-crude oil sources such as those derived from biomass may be added to one or more fuel pools.

It will be understood that a refinery which produces one or more fuel pools, generally with the co-production of other desirable products, is complex and will comprise a plurality of processing units. These refineries generally include a means to carry out the so-called cracking of high-boiling, high-molecular weight hydrocarbon fractions of crude oil to more valuable products including product fuels. Historically the cracking was carried out thermally however it is now conventional to utilise a fluid catalytic cracking process and therefore the refinery will generally include a fluid catalytic cracking unit. In the fluid catalytic cracking unit, the feedstock, which will generally comprise one or more of vacuum gas oils, atmospheric residues and vacuum residues, is contacted with fluidised powdered catalyst such that shorter molecules are formed. The feedstock to the fluid catalytic cracking unit will generally comprise the portion of crude oil having an initial boiling point of at least 320° C. and an average molecular weight ranging from about 200 to 600 or higher. Other processing units to carry out the production of shorter molecules may be used. In one arrangement a deep catalytic cracking unit may be used. This is a specific form of fluid catalytic cracking unit.

Within the various process units within the refinery a substantial amount of light olefins, such as C₂ to C₄ olefins, will be produced. Whilst these olefins could be added to the fuel pools such as the gasoline pool, this can be problematic if done with more than small amounts of the olefins because of the effect on the Reid vapour pressure. This is particularly problematic where ethanol is to be added to the gasoline to meet environmental requirements.

Some of these olefins, in particular ethylene and propylene, can be used as starting materials for the production of useful petrochemicals such as polyethylene and polypropylene and it is therefore desirable, in some situations, to consider recovering one or both of these compounds.

However, the quantity of ethylene produced in the refinery is usually insufficient to justify the costs associated with separating it from the dry gas from the fluid catalytic cracking unit which will generally also include hydrogen, methane, ethane, and some C₃ and C₄ compounds. Thus whilst any ethylene produced may be sold for polyethylene production, it is more usually used as refinery fuel. That is to say the dry gas including the ethylene may be used to produce energy to power the operation of the refinery operation. This may be used in combination with other compounds from within the refinery. Similarly where other cracking means are used waste streams from the cracking unit including ethylene may be uses as refinery fuel. Where a coker unit is present, this too will provide a stream which can be used as refinery fuel.

Propylene is produced in higher quantities and there are therefore economic advantages to recovering it. This may be sold as refinery grade propylene, i.e. without separation of propane or it may be purified to chemical or polymer grade propylene before sale. However, in some circumstances, such as where the refinery is located inland and/or at a remote location, there may be no easy access to the propylene markets. In these circumstances, the propylene can only be sold in combination with propane or in a mixture with an LPG product, where produced in the refinery. Alternatively, the propylene may be used as refinery fuel. However, in these circumstances, its value is reduced not only to below the propylene value but also below the value of transportation fuels.

One proposal for dealing with the presence of propylene is to subject it to dimerization to form a C6 olefin for addition to the fuel pool. However, these generally branched C6 dimers, have a high vapour pressure and therefore, whilst the process enables the propylene to be used in fuel, its addition to the fuel pool can be problematic.

Butene may also be produced within the refinery. In a typical refinery, the C4 olefins may be fed to an alkylation unit where they are transformed with iso-butane into high octane C8 alkylate. However, when the refinery comprises a fluid catalytic cracking unit having a high capacity in relation to the refinery size, the refinery does not always have sufficient iso-butane to enable all of the C4 olefins to be alkylated. Thus, even when an alkylation process is present, there will generally be some residual C4 olefins. One outlet for these C4 olefins would be as fuel gas or the LPG pool.

Where present, the fluid catalytic cracking unit is typically fed with at least part of the vacuum gasoil produced in the refinery. However, it may additionally or alternatively be fed with at least a portion of the atmospheric residue and/or the vacuum reside.

The typical composition of the light components leaving a fluid catalytic cracking unit and deep catalytic cracking unit is set out in Table 1.

TABLE 1

| | Yield weight percent on fluid catalytic |
|---|---|
| | cracking unit (FCCU) or deep catalytic |
| Product | cracking unit (DCCU) |
| Dry gas (which will | 2.5 to 6.0 wt % for FCCU |
| include ethylene) | 4.0 to 10 wt % for DCCU |
| Ethylene | 0.7 to 1.3 wt % mostly as a component of the |
| | dry gas |
| Propane | 1.0 to 2.5 wt % for FCCU |
| Propylene | 3.0 to 7.0 wt % for FCCU |
| | 15 to 25 wt % for DCCU |
| Butenes and total C4 | Butenes for 4 to 12 wt % for FCCU |
| cut (butenes, iso- | Total C4 cut 5.5 to 20 wt % |
| butenes, n-butane and | |
| i-butane) | |

It will therefore be understood that these low carbon alkenes produced in the refinery which are not useful as product fuels cannot readily be added to the fuel pools, and thus they represent a loss of fuel return from a barrel of oil. Whilst these compounds can be used to provide power within the refinery, it is desirable to find an arrangement where they can be converted to product fuels.

It has now been found that by converting the light olefins into alcohols within the refinery, various advantages may be obtained.

Thus according to the present invention there is provided a process for the production of one or more product fuels comprising:
recovering a stream comprising olefin from one or more processing units within an oil refinery;
feeding said olefin stream to a hydroformylation zone within the refinery;
contacting said olefin stream with carbon monoxide and hydrogen in the presence of a hydroformylation catalyst and operating said hydroformylation zone under hydroformylation conditions such that at least a portion of the olefin is converted to an aldehyde having an additional carbon atom to the olefin;
recovering a stream comprising said aldehyde from the hydroformylation zone and passing said aldehyde stream to a hydrogenation zone operated under hydrogenation conditions such that at least a portion of the aldehyde is converted to the corresponding alcohol; and
recovering a stream comprising alcohol and forwarding the alcohol to one or more product fuel pools within the refinery.

Thus in one arrangement, the process of the present invention enables product fuels, such as motor fuels, from, for example, propylene which will be contained in a low purity gas within a refinery.

Alkanes formed within the process may additionally be recovered and forwarded to one or more fuel pools within the refinery.

The stream comprising the olefin may be recovered from any suitable processing unit within the refinery. It may be recovered from two or more processing units within the refinery. In this arrangement, the streams from the separate processing units may be combined before they are fed to the hydroformylation zone or they may be fed separately thereto.

In one arrangement, the stream comprising the olefin may comprise a stream recovered from a fluid catalytic cracking unit.

The stream recovered from the one or more processing units may be fed directly to the hydroformylation zone or it may be subjected to further processing first. These processes may include the removal of impurities in the feed that will affect the operation of the hydroformylation catalyst. These impurities may include one or more of dienes, acetylenes, hydrogen sulphide, sulphur-containing compounds such as mercaptans and thiophenes, and metal carbonyls.

The stream comprising olefin will generally comprise lower olefins and will generally be a mixture of olefins. The stream will generally comprise a mixture of C₂ to C₅ olefins. It will be understood that olefins having a higher number of carbon atoms may be used. However, the higher olefins are generally directed to the appropriate fuel pool without requiring processing to increase the number of carbons present. The stream may comprise a single olefin but generally it may be a mixture of olefins.

The stream comprising olefin will generally comprise from about 5 to about 95% olefin. The other components present will depend on the source of the stream but may include hydrogen, alkanes, and other olefins. Generally it will be desirable for the stream comprising olefin to have a high concentration of olefin in order to maximise the efficiency of the processing of the stream and/or to minimise the size of equipment required. Thus the stream may comprise at least about 50% olefin and may have more than about 60% or more than about about 70% olefin.

Where ethylene is present in the stream fed to the hydroformylation zone, propanal will be formed. Hydrogenation of propanal gives 1-propanol, which can be blended into the gasoline pool as its Research Octane Number (RON) is 118 and its Motor Octane Number (MON) is 98. Hydroformylation of propylene yields a mixture of iso- and normal-butyraldehyde, which after hydrogenation forms iso- and normal-butanol. The RON of iso-butanol is 105 and its MON is 93, while the RON of normal-butanol is 98 and its MON is 85.

It will be understood, that within the refinery, streams are generally cuts from the distillation downstream of crackers, for example, a so-called C3 cut, whilst comprising substantially propylene will also contain small amounts of iso and n-butenes. These butenes will be transformed into valeraldehyde or isomers of valeraldehyde that will then be hydrogenated into pentanols.

The alcohols produced are generally suitable for addition to the gasoline pool. Some may also be suitable for addition to other fuel pools. Thus the process of the present invention enables compounds which would otherwise be lost or simply burnt as fuel for the refinery to be added to the fuel pools thereby increasing the efficiency of the refinery and maximising the fuel production thereof.

Where the stream comprising olefin comprises propylene, the product of the hydroformylation process and hydrogenation process will generally include a mixture of iso-butanol and n-butanol. The ratio of iso-butanol to n-butanol will generally vary depending on the catalyst and operating conditions used. However, it will generally be between about 50 to about 3 wt % iso-butanol and about 5 to about 97 wt % n-butanol. It will therefore be understood that most of the above mixtures of iso-butanol and N-butanol may be mixed to the gasoline pool without penalty or, with very small penalty, on the gasoline octane constrained as far as the gasoline octane specification is concerned.

Where the stream comprising olefin comprises C5 olefins, these can be used to form hexanol. Whilst hexanol may not be a useful addition to the gasoline pool, it can be transferred to hexane which can be added to the gasoline pool. This would be particularly advantageous as it will not have adverse Reid vapour pressure implications. Some of all of the C6 aldehyde could be sent to aldolisation to make C12 paraffin which can be added to the diesel pool.

Any suitable hydroformylation process may be carried out in the hydroformylation zone. Whilst a standalone hydroformylation plant will generally be designed to convert a single olefin into its corresponding aldehyde, the stream fed to the hydroformylation zone in the process of the present invention, will generally be required to convert olefins of different length. Whilst separate hydroformylation process units may be used for different olefins, in one arrangement, a single process unit may be used although it will be understood that more than one reactor may be used within the process unit.

In one arrangement, an olefin stream having a first number of carbon atoms and an olefin stream having a second number of carbon atoms may be co-fed and co-processed in the same reactor or series of reactors. Thus for example, an ethylene-containing stream and a propylene-containing stream may be co-processed in the same reactor or series of reactor.

In one alternative arrangement, an olefin stream having a first number of carbon atoms is fed to a first reactor within the hydroformylation zone where it is reacted with carbon monoxide and hydrogen so that at least a portion of the olefin is converted to the corresponding aldehyde. The product stream from this reactor is then passed to a second reactor to which an olefin stream having a second number of carbon atoms is also fed. In this second reactor at least a portion of the olefin stream having a second number of carbon atoms is converted to the corresponding aldehyde. In addition, reaction of unreacted olefin having a first number of carbon atoms may also occur. In this arrangement, additional carbon monoxide and hydrogen may be added to the second reactor. Thus, for example, a stream comprising propylene, generally in mixture with other components, may be fed to a first reactor. The liquid product from this reactor which will comprise butyraldehydes, catalyst solution, unreacted olefins and other dissolved gases is passed to a second reactor. A stream comprising ethylene is then fed to the second reactor. This stream may be from the same source as that fed to the first reactor or a different source. The propylene may be that recovered from a fluid catalytic cracking unit, usually in the dry gas.

In a second alternative arrangement the hydroformylation of an olefin stream having a first number of carbon atoms and an olefin stream having a second number of carbon atoms is carried out in parallel. Any suitable arrangement may be used. One example of a suitable arrangement can be found in WO2015/094781 the contents of which are incorporated herein by reference. Thus, for example, refinery grade propylene may be fed to one reactor and ethylene may be fed to a second reactor operating in parallel. Where a parallel arrangement of reactors is used, the product streams thereof may be combined before being passed to the hydrogenation zone.

Whilst the above alternatives have been discussed in connection with recording treating streams comprising ethylene and propylene, it will be understood that similar arrangements may be used for other olefin comprising streams having different number of carbon atoms.

Whatever process is used, any suitable catalyst may be used. The catalyst will generally be selected to optimise the hydroformylation of the olefin present in the feed. The most active catalysts for the hydroformylation of propylene are homogenous solutions of rhodium-ligand complexes. The ligand used can strongly influence the catalyst activity and the normal/iso ratio obtained. Examples of suitable ligands include phosphine type ligands such as astriphenylphosphine and cyclohexanediphenylphosphine, monophosphite ligands such as tris(2,4-di-tert-butylphenyl)phosphite, bisphosphite ligands, and polyphosphite ligands.

Where a plurality of reactors is used, it will be understood that they may use the same or different catalysts. Similarly, they may operate under the same, or different, reaction conditions.

The carbon monoxide and hydrogen fed to the hydroformylation zone may be provided from within the refinery thereby further integrating the hydroformylation reaction and improving the economics. The carbon monoxide and hydrogen synthesis gas may be produced by steam reforming.

As the feed to the hydroformylation zone may be the dry gas from the cracking unit, it will generally include hydrogen and thus the amount of hydrogen which has to be added to the hydroformylation zone can be reduced.

Additionally or alternatively, the carbon monoxide, hydrogen may be produced from bio-sources and as such its use in the process of the present invention will offer cost advantages and improve the bio content of the fuel pool. This may enable the amount of ethanol which is currently added to the fuel pool to meet bio-source requirements to be reduced. This is beneficial as ethanol is expensive and raises issues with the Reid vapour pressure specifications of the gasoline, especially in hot weather conditions.

Once hydroformylation has been carried out, the stream comprising the aldehyde is recovered from the hydroformylation zone and passed to the hydrogenation zone. Prior to being passed to the hydrogenation zone, the stream comprising the aldehyde may be passed to a recovery section, where any dissolved gases are removed, and the catalyst solution may be recovered. Where the aldehyde containing stream comprises more than one aldehyde, these may be separated prior to hydrogenation, or they may be hydrogenated is a single feed.

Alkane present may be removed before the aldehyde is passed to the hydrogenation zone.

Where the olefin stream comprises propylene such that butanol will be formed, as iso-butanol has a higher RON and MON than n-butanol, it is preferable to produce an n/i-butanol mixture that contains as much iso-butanol as possible. In this arrangement, the conditions used for the hydroformylation, for example, the hydrogen and carbon monoxide partial pressures and catalyst or selected so that the ratio of iso-butanol to n-butanol in the mixture contains a maximum content of iso-butanol are all of the butanol produced is to be fed to a gasoline pool.

The catalyst and/or the process conditions may be selected to maximise the formation of the branched aldehydes for the propylene and any butene in the feeds as the equivalent alcohols are preferred as gasoline additives.

In an alternative arrangement, the n-butanol and iso-butanol may be separated, generally by distillation, and sent to different fuel pools. For example, iso-butanol may be sent to a high octane fuel pool and n-butanol may be sent to a low octane fuel pool. In an alternative, iso-butanol may be sent to a low octane fuel pool to lift the average octane number and the n-butanol may be fed to a fuel pool which requires less or no good increase in octane number. In an alternative arrangement, only one of the butanol may be sent to a fuel pool and the other may be separated for a different use. For example, the n-butanol, iso-butanol or both n-butanol and iso-butanol may be separated and commercialised as a petrochemical or as a solvent.

Alternatively, the iso-butyraldehyde and n-butyraldehyde may be separated, generally by distillation, before hydrogenation. Hydrogenation of the iso- and normal-butyraldehyde separately will generate iso-free normal-butanol and normal-free iso-butanol. Separate hydrogenation zones may be provided for the iso- and normal-butyraldehyde. Alternatively, one of the isomers may be temporarily stored whilst the other is processed through hydrogenation refining. As an alternative to storing one of the isomers, it may be sent elsewhere within the refinery for treatment.

Where C4 olefins are present within the refinery, as an alternative to the conventional alkylation thereof, they may be treated according to the process of the present invention. It will therefore be understood that the alkylation may be replaced with the process of the present invention or both processes may be available such that some C4 olefins are treated via alkylation and some via the hydroformylation and hydrogenation of the present invention. Where both are present, the user will be able to alter the flow of C4 olefins between the two systems to meet demand, or to take account of fluctuations in for example, the iso-butane required by the alkylation unit.

Where C4 olefins are subjected to the hydroformylation and hydrogenation of the present invention a valeraldehyde product may be obtained in the hydroformylation zone. The valeraldehyde cut may be separated and fed to an aldolisation unit for conversion to C10 enals. These enals may then be hydrogenated in the hydrogenation zone to saturated alcohols, which could be added to an appropriate fuel pool or could be further processed to the corresponding alkane.

This process may enable a mono-branched, low density and high octane product to be produced which will a useful addition to the diesel pool and will be a good aviation fuel.

Any suitable hydrogenation process may be carried out in the hydrogenation zone. Any suitable catalysts may be used. Process conditions will be selected depending on the composition of the feed to the hydrogenation zone and the catalyst being used.

The stream recovered from the hydrogenation zone will comprise alcohol corresponding to the, or each, aldehyde present in the feed to the hydrogenation zone. This stream will generally be treated to separate the alcohol from other components in the stream. This separation will generally be carried out by distillation.

As the process of the present invention enables olefins having a low number of carbon atoms to be reduced into compounds which may be included within one or more fuel pools, a significant number of advantages are achieved. For example, in some territories, it is now desirable that fuels contain a proportion of alcohol. As higher alcohols, such as butanol, have a higher calorific value and a lower vapour pressure than ethanol, their addition to the fuel pool offers the opportunity to find an improved fuel.

The process of the present invention is particularly useful for the production of gasoline. However, the alcohols produced could also be added to other fuel pools, such as the diesel pool.

It will therefore be understood, that the process of the present invention which integrates the treatement of the low carbon olefins within the refinery may be operated to enable refinery operators to maximise the output of the refinery. In particular it will enable molecules which in prior art arrangements could not effectively be added to the fuel pool to be converted efficiently to molecules which can be added to the fuel pool and thereby add to the useful product fuels which can be recovered from the crude oil.

In addition, the process enables the operator to tune the alcohols being achieved and their addition to one or more fuel pools. Thus the operation of the hydroformylation zone and the hydrogenation zone may be manipulated to take account of changes in the feedstock the refinery, the performance of processing units within the refinery and the like. Prior to the present invention operators of refineries, who could not find a market for propylene or butenes, were forced to operate the refinery to minimise the amount of propylene or butenes produced. This could negatively impact on the performance of the refinery. Since the present invention enables the propylene to be converted to butanol, which can be used in the fuel pool, such as the gasoline fuel pool, such considerations do not have to be taken into account as the production of propylene enables an increase in gasoline product. Similar benefits are noted for the other olefins.

A further advantage of the present invention is that since the product of the hydroformylation and hydrogenation zones is to be added to the fuel pool, the purity levels required for the alcohols is less than that required where they are for other uses. Thus, for example, where iso-butanol is to be used as a solvent, it must have a higher purity than when the iso-butanol is to be added to the gasoline pool. It is therefore possible to operate with smaller, or less rigorous refining procedures.

Additionally, or alternatively, catalysts can be used for a longer period. In this connection, it will be understood that, for example, hydrogenation catalysts tend to generate more side products as they age. This would be permissible in, for example, the production of butanol, which is to be added to fuel but would generally not be acceptable in the production of chemical grade butanols.

Similarly, catalysts used in the hydroformylation reaction will generally lead to the formation of heavies which where the product is for fuel use is less problematic than in other arrangements. This is because the heavies produced may be passed directly to the fuel pool or they may be recycled to suitable processing units within the refinery such as the fluid catalytic cracker.

The purity requirements of the olefin stream recovered from the one or more processing units may be reduced when compared to those required for other processes. This will particularly apply to impurities which contribute to heavies formation such as iron.

It should be noted that one aspect of the proposed flowsheet is that any increased heavies make could be recycled to a suitable unit such as the fluid catalytic cracking unit or to a hydrocracker and hence the heavies made would not be a loss on the feedstock efficiency.

As any increased heavies make can be utilised or recovered it may be possible to use carbon steel as the material of construction for the hydroformylation reactors rather than the conventional stainless steel used in chemical grade hydroformylation plants.

The process of the present invention will now be described with reference to the accompanying drawings in which:

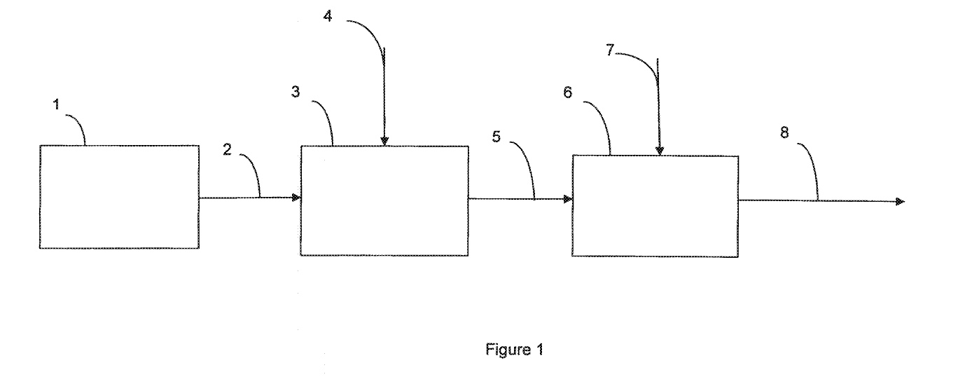

FIG. 1 is a schematic representation of the process of the present invention;

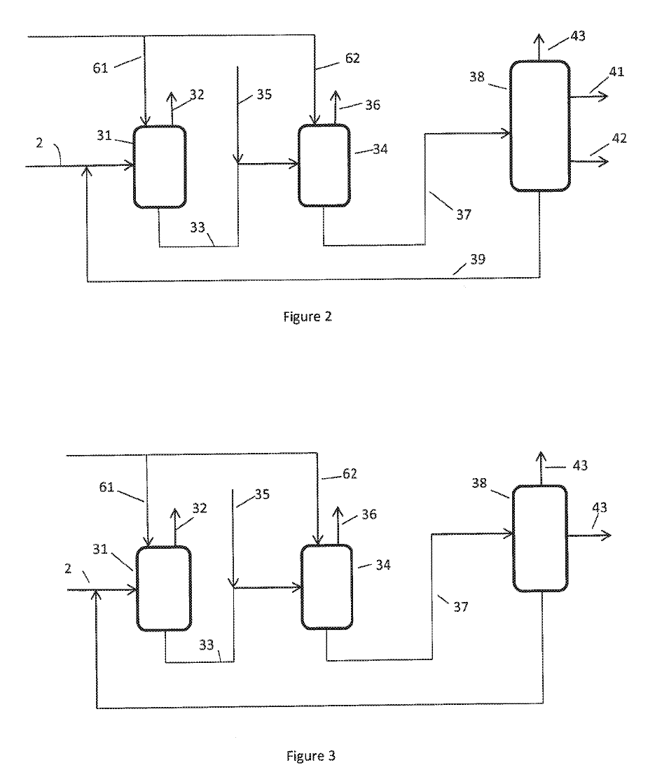

FIG. 2 is one example of an arrangement for the hydroformylation zone of the present invention;

FIG. 3 is a second example of an arrangement for the hydroformylation zone of the present invention; and

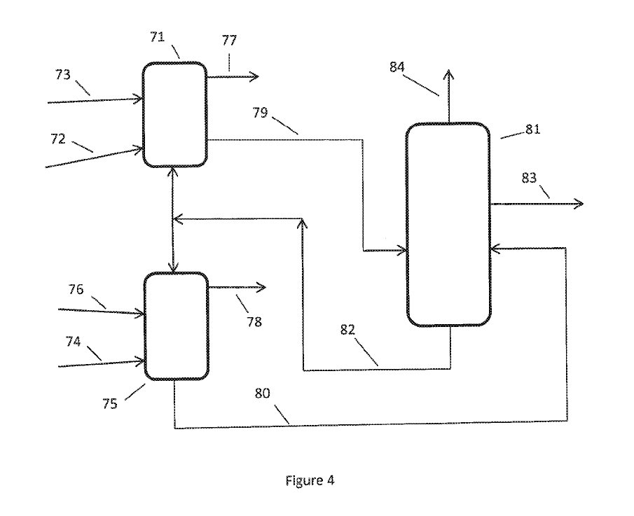

FIG. 4 is a third example of an arrangement for the hydroformylation zone of the present invention.

It will be understood that the drawing is diagrammatic and that further items of equipment such as drums, pumps, sensors, valves, controllers, holding tanks, storage tanks and the like may be required in a commercial plant. The provision of such ancillary items of equipment forms no part of the present invention and is in accordance with conventional chemical engineering practice.

For convenience, the present invention will be described with reference to the treatment of ethylene and/or propylene. However, it will be understood that it applies equally to other processes.

A stream 2 is recovered from a processing unit 1. This is passed to a hydroformylation zone 3 where the olefin is contacted with carbon monoxide and hydrogen added in line 4 in the presence of a suitable catalyst. At least a portion of the olefin is converted in the hydroformylation zone 3 to an aldehyde having one more carbon that the olefin.

A stream comprising the aldehyde is recovered in line 5 and passed to a hydrogenation zone 6 where it is contacted with hydrogen added in line 7 such that hydrogenation occurs in the presence of a suitable catalyst to form the corresponding alcohol. A stream 8 comprising the alcohol is recovered and passed to the fuel pool within the refinery.

The hydroformylation zone 3 may comprise two reactors. One arrangement is illustrated in FIG. 2. In this arrangement, the stream 2 is passed to a first hydroformylation reactor 31 where it is contacted with a carbon monoxide and hydrogen gas added in line 61. A portion of the olefin present in the stream 2 will be converted to the corresponding aldehyde. A vent 32 will be provided to the reactor.

A stream 33 comprising unreacted olefin and reacted aldehyde is passed to a second hydroformylation reactor 34 where it is contacted with further carbon monoxide and hydrogen gas added in line 62. Additional olefin comprising feed may be added in line 35. Further reaction will occur in the second hydroformylation reactor 34 such that further aldehyde is produced. A vent 36 will be provided to the reactor.

A stream 37 comprising the aldehyde is recovered from the second hydroformylation reactor 34 and fed to a separation unit 38. Separated catalyst is recycled to the first hydroformylation reactor in line 39. Light aldehyde product is recovered in line 41 and heavier aldehyde product in line 42. A vent stream is removed in line 43.

An alternative arrangement is illustrated in FIG. 3. This is the same as that illustrated in FIG. 2 except that the light and heavy aldehydes are not separated in the separation unit 38 such that there is a single stream 44 recovered therefrom.

A still further arrangement is illustrated in FIG. 4. In this arrangement, the two hydroformylation reactors are operated in parallel. For example, a dry gas feed 73 is fed to a first hydroformylation reactor 71 where it is contacted with carbon monoxide and hydrogen added in line 72 where reaction of at least some of the olefin present is converted to the corresponding aldehyde. A vent 77 is provided on the hydroformylation reactor 71. A second feed 74, such as a refinery grade olefin feed, is fed to a second hydroformylation reactor 75 where it is contacted with carbon monoxide and hydrogen added in line 76 where reaction of at least some of the olefin present is converted to the corresponding aldehyde. A vent 78 is provided on the hydroformylation reactor 75.

Streams 79 and 80 comprising the corresponding aldehyde are fed to the separation unit 81 where it separated from the catalyst which is recycled in line 82 to the hydroformylation reactors. A mixed aldehyde product is recovered from the separation unit 81 in line 83 to be fed to hydrogenation. The separation unit 81 includes a vent 84.

Whatever arrangement is used, suitable operating conditions will be selected. These will depend on the feed, the catalyst and the like. Generally the hydroformyation will be carried out at a temperature of from about 70 to about 110° C. and at pressures of from about 200 to about 260 psi.

1. A process for the production of one or more product fuels comprising:
recovering a stream comprising olefin from one or more processing units within an oil refinery;
feeding said olefin stream to a hydroformylation zone within the refinery;
contacting said olefin stream with carbon monoxide and hydrogen in the presence of a hydroformylation catalyst and operating said hydroformylation zone under hydroformylation conditions such that at least a portion of the olefin is converted to an aldehyde having an additional carbon atom to the olefin;
recovering a stream comprising said aldehyde from the hydroformylation zone and passing said aldehyde stream to a hydrogenation zone operated under hydrogenation conditions such that at least a portion of the aldehyde is converted to the corresponding alcohol; and
recovering a stream comprising alcohol and forwarding the alcohol to one or more product fuel pools within the refinery.

2. The process according to claim 1, wherein alkanes formed within the process are additionally recovered and forwarded to one or more fuel pools within the refinery.

3. The process according to claim 1, wherein the stream comprising the olefin may comprise a stream recovered from a fluid catalytic cracking unit.

4. The process according to claim 1, wherein the stream comprising olefin comprises a mixture of olefins.

5. The process according to claim 1, wherein the stream comprising olefin comprises a mixture of C₂ to C₅ olefins.

6. The process according to claim 1, wherein the stream comprising olefin comprises a mixture of C₅ to C₆ olefins.

7. The process according to claim 1, wherein the alcohol recovered will be suitable for addition to the gasoline pool.
